(19) United States
(12) Patent Application Publication
(10) Pub. No.: US 2025/0392644 A1
(43) Pub. Date: Dec. 25, 2025

(54) METHODS AND SYSTEM FOR AUTOMATIC JOINING AND INFORMATION EXCHANGE IN A MESH NETWORK

(71) Applicant: Hill-Rom Services, Inc., Batesville, IN (US)

(72) Inventors: Jason M Williams, Batesville, IN (US); Jaclynn Siler-Dearring, Batesville, IN (US)

(73) Assignee: Hill-Rom Services, Inc., Batesville, IN (US)

(21) Appl. No.: 19/248,265

(22) Filed: Jun. 24, 2025

Related U.S. Application Data
(60) Provisional application No. 63/663,973, filed on Jun. 25, 2024.

(51) Int. Cl. H04L 67/12; G16H 40/63; H04W 4/029

(52) U.S. Cl. CPC H04L 67/12; G16H 40/63; H04W 4/029

(57) ABSTRACT

Methods and systems for the automatic addition and removal of medical devices to a localized area network such as a mesh network and the detection of tagged devices and caregivers entering and leaving a patient area. Detection of medical devices and caregivers may trigger the execution of predefined workflows by the devices connected to the localized area network. Recognition and initiation of actions may be based in part on the presence of a localized area network of devices plugged into smart outlets.

### CROSS-REFERENCE TO RELATED APPLICATION

This application claims priority to and the benefit of the earlier filing of U.S. Provisional Application No. 63/663,973, filed on Jun. 25, 2024, which is incorporated by reference herein in its entirety.

### TECHNICAL FIELD

This application relates generally to automatically connecting devices to a localized mesh network and relaying information among devices in the mesh network and to a real time location system (RTLS).

### BACKGROUND

In the United States, there are an average of 15-20 medical devices per hospital room. As multiple people and multiple devices move in and out of patient rooms, it becomes challenging to associate the people and devices with the specific patient or patient room, impacting the security of the devices, the quality and amount of the information that may be obtained, and the responsiveness of caregivers.

The increasing number of devices in patient settings compounds the demands placed on caregivers leading to delayed response times and potential medical errors. Information coordination between devices and caregivers, and increased automation, reduces the burden on caregivers, thereby improving patient care and satisfaction.

### SUMMARY

Provided are methods and systems for the automatic connection of medical devices to a many-to-many localized area network topology (mesh network). Such systems may include one or more medical devices connected to respective smart outlets, including receptacles, with connectivity capabilities. The medical devices may be plugged into the smart outlets via a wired electric cord and the smart outlets and medical devices may form a mesh network allowing for communication between the devices and the outlets. In some aspects, one or more of the devices and/or outlets may be in communication with a server in communication with a real-time location system (RTLS).

In some aspects, additional medical device(s) may be brought into the patient environment or area and plugged into a respective smart outlet. By plugging the medical device into the smart outlet, the additional medical device(s) may automatically join the existing mesh network. Such an existing network may be made up of smart outlets or a combination of smart outlets and devices. Information may be transmitted among the medical devices and smart outlets and/or between one or more of the smart outlets and medical devices and the server connected to the RTLS system. In some aspects, each medical device or smart outlet may be able to communicate directly with the server. In other aspects, only one or some of the medical devices and/or smart outlets may be able to communicate directly with the server.

In some aspects, an RTLS tag may be attached to a device or a caregiver. Each of the server, RTLS anchor, and one of more of the medical devices may contain at least one processor such that the processor of the RTLS anchor is configured to track a location of an RTLS tag and communicate the position to the RTLS. The RTLS may be configured by the RTLS processor to communicate the position of the tag received from the RTLS anchor with the server and the server may be configured to communicate information regarding the tag. The server may transmit the location of the RTLS tag (and the attached device or caregiver by proxy) to one or more of the medical devices and/or outlets and the medical device and/or outlet that has received the location information may further communicate such information to one or more of the other medical devices and/or outlets in a patient area.

In some aspects, information regarding the RTLS tag may be used to provide varying levels of access to the medical devices in the patient area. For example, an RTLS tag may be associated with a caregiver who has authorization to resolve alarms on a medical device but not change the programming of the medical device. Based on the RTLS tag, the system may therefore prevent alteration of the programming of the medical device. In some aspects, an RTLS tag may allow different levels of access for different medical devices within a patient's room. Other tags associated with other caregivers may give more or less access to one or more of the networked medical devices or the system overall. In some aspects, when a medical device is notified by the server of the presence of a specific tag, information regarding the presence of the specific tag may be transmitted to the other medical device(s) and/or smart outlets within the patient area. Each medical device may then identify the action to be taken or the program to be executed, if any, based on the presence of the specific tag. In some aspects, there may be actions to be taken in the absence of a specific tag such that when the location of the specific tag changes, each medical device may identify an action to be taken. For example, when an RTLS tag associated with a caregiver exits a room, alarms may be re-set, or restrictions may be enacted by the various medical devices.

To the accomplishment of the foregoing and related ends, certain illustrative aspects of the system are described herein in connection with the following description and the attached drawings. This summary is provided to introduce a selection of concepts in a simplified form that are further described below in the Detailed Description. This summary is not intended to identify key features or essential features of any subject matter described herein.

### DESCRIPTION OF THE FIGURES

The following figures, which form a part of this disclosure, are illustrative of the described technology and are not meant to limit the scope of the claims in any manner.

FIG. 1 illustrates a schematic block diagram of an example hospital system according to an embodiment.

FIG. 2 illustrates a medical device and system connections within a patient environment according to an embodiment.

FIG. 3 illustrates medical device and system connections within a patient environment according to an embodiment.

FIG. 4 illustrates the addition of a device to the patient environment of FIG. 3.

FIG. 5 illustrates the integration of a new medical device into a group of networked devices within a patient environment according to an embodiment.

FIG. 6 illustrates the entrance of a caregiver into the patient environment of FIG. 5.

FIG. 7 illustrates medical device connections within a multi-patient environment according to an embodiment.

FIG. 8 illustrates a method for pairing a medical device to a mesh network according to an embodiment.

FIG. 9 illustrates a method for detecting an RTLS tag and associating the RTLS tag with a mesh network according to an embodiment.

FIG. 10 illustrates a method for detecting the departure of an object from a networked environment according to an embodiment.

FIG. 11 is a schematic diagram of a system for automatically provisioning devices in a localized wireless network according to an embodiment.

FIG. 12 is a schematic diagram of a system for automatically provisioning devices in a localized wireless network according to an embodiment.

FIG. 13 is a block diagram of a computing system, according to at least one example.

### DETAILED DESCRIPTION

Various implementations of the present disclosure will be described in detail with reference to the drawings, wherein like reference numerals present like parts and assemblies throughout the several views. Additionally, any examples set forth in this specification are not intended to be limiting and merely set forth some of the many possible implementations.

Provided are methods and systems for the automatic connection of medical devices to a many-to-many localized area network topology (mesh network). In some aspects, such a mesh network may be a Bluetooth mesh network using Bluetooth Low Energy protocol as described at Bluetooth.com, formed through devices connected via a wired electric plug to outlets, including receptacles, with connectivity capabilities (smart outlets), allowing the devices to communicate with each other, the outlets, and in some embodiments with a central server or a real-time location system (RTLS), even if some of the devices, outlets, and servers are not within direct range. In some aspects, the devices and outlets may relay messages through other devices in the network. In some aspects, such methods and systems may trigger automatic workflows and actions upon the entrance or departure of RTLS tagged caregivers and/or medical devices, as well as other devices, into a particular patient environment such as a patient room. In situations where there is more than one patient in a patient area such as a room, smart outlets and the devices plugged into them may form one or more clusters where a set of devices associated with one patient or one portion of the room associated within the cluster may communicate with other devices within that cluster, but does not communicate across clusters with a second set of devices. In some aspects, the smart outlets automatically form a mesh network and connect devices to the mesh network when the devices are plugged into the smart outlets via a wired electric cord.

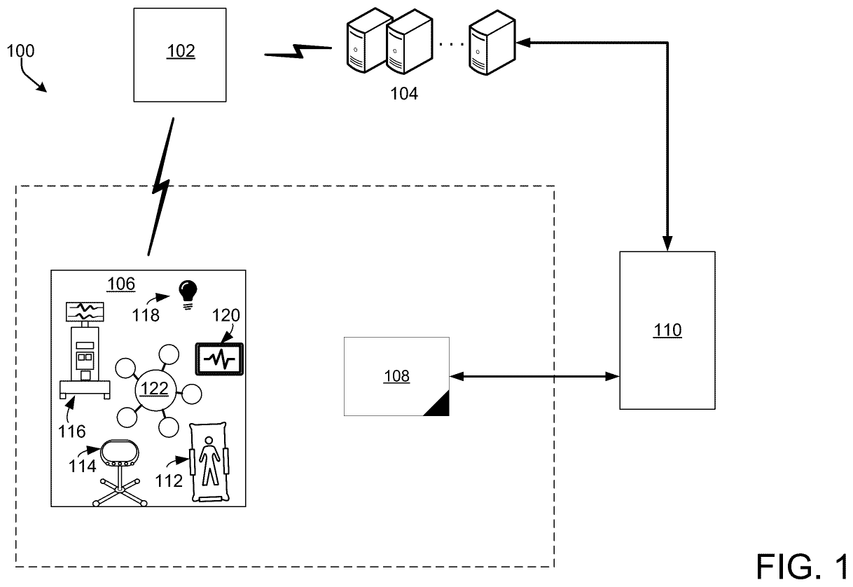

FIGS. 1-7 illustrate a hospital room with various configurations of devices, patients, and caregivers. Each of FIGS. 1-7 may apply to the same room at different points in time or different rooms at the same or different points of time. For ease of understanding, the numbering of the devices and the connections between the devices is maintained across the figures, though such numbering is not intended to limit the invention and one of ordinary skill in the art would understand that similar arrangements would apply to the same or different devices, patients, and caregivers.

FIG. 1 depicts an example healthcare environment 100 including a server 102, a real time location system (RTLS) 104, a RTLS anchor 110, a RTLS tag 108, and a set of exemplary networked devices within a patient area 106. Exemplary networked devices include, for example, a patient bed 112, a free-standing monitor 114, an infusion pump 116, a lighting system 118, and an EKG 120 though additional or fewer devices are also contemplated.

One or more medical devices including a patient bed 112, a free-standing monitor 114, an infusion pump 116, a lighting system 118, and an EKG 120 may be connected to each other through a mesh network 122. Each networked device may include a device communication module that is configured to communicate with an outlet communication module in a smart outlet such that the networked device(s) and the outlet(s) can share data wirelessly. Once a device is added to the mesh network, the device communication module of the first networked device is enabled to communicate with the device communication module of a second networked device or outlet through the mesh network, thus, after the device is joined to the mesh network via the smart outlet, the device may communicate directly with other networked devices and outlets that are part of the mesh network. Some device communication modules may allow for communication with the server 102 and/or the RTLS 104 using, for example, a Wi-Fi protocol or other communication protocol including Bluetooth, near field communication (NFC), ZigBee, Bluetooth, Z-wave, 6LoWAPAN, Wi-Fi, cellular technologies, local area networks, LoRa, NB-IT, and the like.

The server 102 and RTLS 104 may or may not be integrated into a larger system within the hospital. The server 102 and RTLS 104 may be the same or different devices and may be located in the same or different remote or local locations. In some aspects, the server 102 and the RTLS 104 may be located in the cloud.

The use of a mesh network creates multiple routes for information to travel among connected nodes. This may increase the resiliency of the network by adding redundancies and allowing for the continuation of patient care, monitoring, and reporting even if a portion of the communication system fails. In the networked environment, one or more of the networked devices may be plugged into a smart outlet, allowing the networked devices to communicate directly with each other or with the smart outlets. In some aspects, the networked devices may communicate localized information directly with each other without going to an outside server or network. In some aspects, one or more of the networked devices may be able to transmit information to or from the server 102 and/or the RTLS 104. Such transmission may be information related to the transmitting networked device or information received by the transmitting networked device from one or more other networked devices. For example, the infusion pump 116 may sound an alarm. Information regarding the alarm may be communicated to the other networked devices within the mesh network 122 such as the bed 112 and/or relayed further along the network to the proper connected recipient such as the server 102 or the stand alone monitor 114. That is information may be sent from the infusion pump 116 to the bed 112 and from the bed 112 to the server 102. In other aspects, the information may be sent from the infusion pump 116 to the bed 112 and from the bed 112 to the monitor 114. In other aspects, the alarm information may be sent from the infusion pump 116 directly to the monitor 114. In some aspects, the alarm information may be sent from the infusion pump 116 directly to the server 102. In some aspects, the communication is targeted, that is, the infusion pump 116 communicates directly with the bed 112 but not with any other devices or servers. In other aspects, the information is broadcast, that is, the infusion pump 116 sends information generally to the other networked devices in the room. In some aspects, one or more of the networked devices such as the bed 112 or the infusion pump 116 may transmit information regarding the alarm to the server 102. In some aspects, receipt of the alarm by the one or more networked devices in the mesh network may initiate action by one or more of the devices on the mesh network 122. For example, an alarm from one device may result in another networked device pausing, starting, or displaying information.

In some aspects, the use of smart outlets may allow for selective delivery of information. Further, it may allow different communication protocols to exist on different devices as it is not necessary that all of the devices be Wi-Fi capable. This may assist in creating a system that is scalable and extensible, allowing for the addition of new functionality and devices without having to redo every part of the system. By using loosely coupled devices, the system described herein may allow for a standard, lower cost way to integrate new devices into an existing network and allow for the localized exchange of information between devices.

The mesh network may also receive and act on information regarding an RTLS tag associated with a device or person. For example, a device or person attached to tag 108 may be identified by an RTLS anchor 110. Such RTLS anchors 110 may be located throughout the home or care facility. Communication between the tag 108 and the RTLS anchors may be through near field communication (NFC), ZigBee, Bluetooth, Z-wave, 6LoWPAN, Wi-Fi, cellular technologies, local area networks, LoRa, NB-IT, and the like. Information regarding the RTLS tag 108 associated with a device or person may be sent through the RTLS to one or more of the devices in the mesh network 122 using one or more communication protocols. Once information regarding the tag 108 associated with the device or person is received by the one or more devices in the mesh network 122, the information may be distributed to the other devices in the mesh network 122. Thus, if infusion pump 116 is able to communicate with the server 102, information regarding the tag 108 associated with the device or person may be sent to the infusion pump 116 and the infusion pump 116 may transmit the information regarding the tag 108 associated with the device or person to the other devices in the mesh network 122 such as the bed 112, free-standing monitor 114, lighting system 118, and EKG 120. In this way, even though communication outside the mesh network was only to one device, the information received by the one device from the RTLS is propagated throughout the mesh network 122.

In some aspects, different tags may grant different privileges. For example, a particular caregiver may have authorization to respond to some alarms or input instructions to some but not all of the networked devices in a patient area. Other caregivers may have authorization to interact with all of the networked devices. The RTLS 104 may inform devices on the mesh network 122 of the identity and/or access level of the caregiver or device associated with the tag, and the networked devices may determine application specific actions or status changes based on the identity of the caregiver that has been shared by the devices across the mesh network 122.

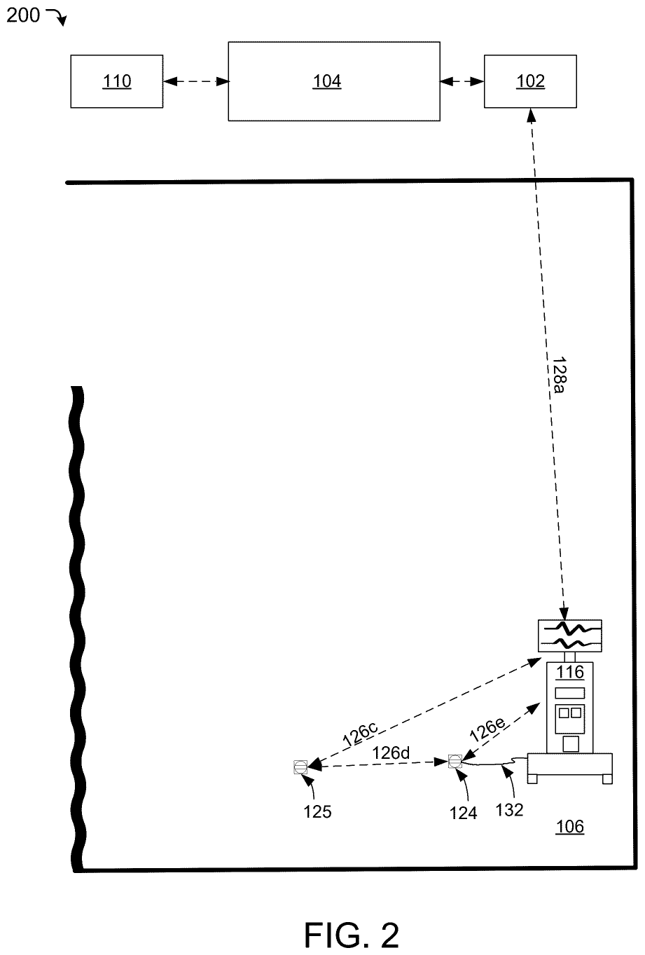

FIG. 2 depicts an environment 200 in which a single device, networked infusion pump 116, has been placed. Networked infusion pump 116, as shown in patient area 106, is able to exchange information via the mesh network shown by 126c, 126d, and 126e with smart outlet 124, which is connected to the infusion pump 116 via wired electric cord 132. While one device and two smart outlets are shown in FIG. 2, there is no restriction on the number of devices, RTLS tags, or smart outlets that may be present in the patient area.

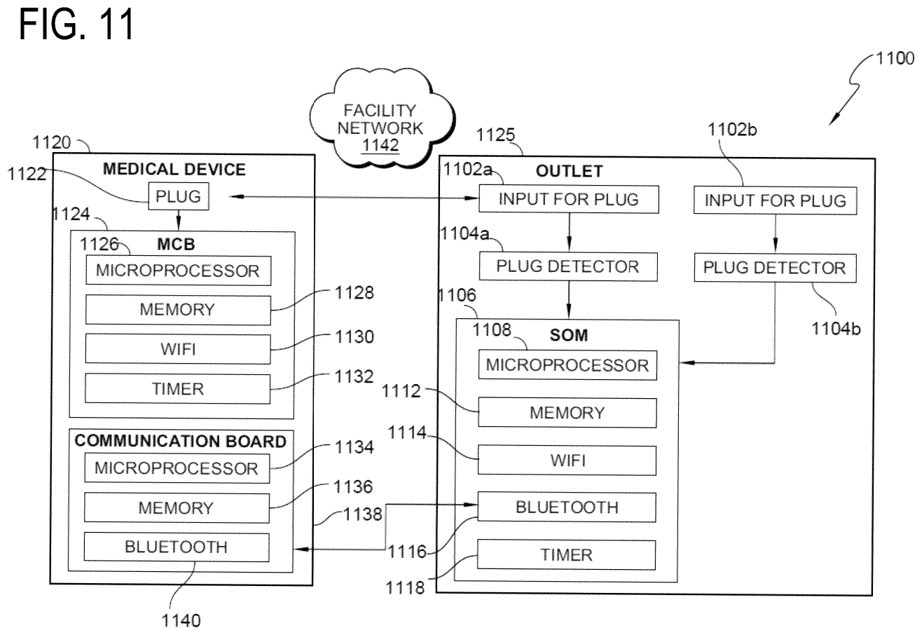

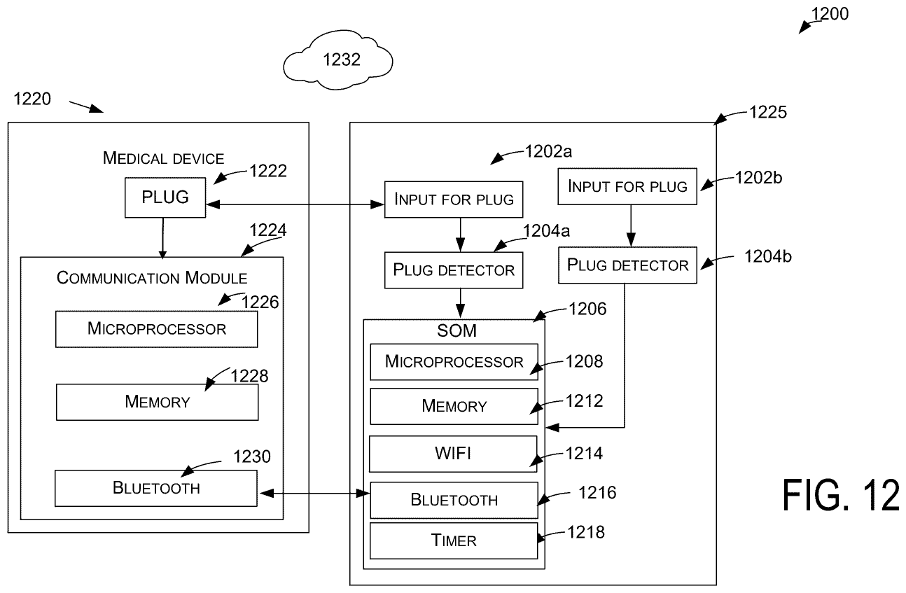

Smart outlets may have a variety of configurations that allow them to detect that an object has been plugged in and to determine if the object that has been plugged in is capable of joining a mesh network. While smart outlets may have a variety of configurations, in some aspects smart outlets include a circuit board and an outlet processor configured to carry out instructions stored in an outlet memory as shown in further detail in FIGS. 11 and 12. A wireless module enables the outlet processor to communicate with and share data with the mesh network as shown at 126c, 126d, and 126e. The smart outlet(s) 124 and 125 may include a power receptacle for a wired electric plug and a plug detector associated with each power receptacle. In some aspects, one or more of the smart outlets may have an indicator that indicates when the outlet communication module is connected to a localized network. Other indicators or types of indicators may be used for other purposes.

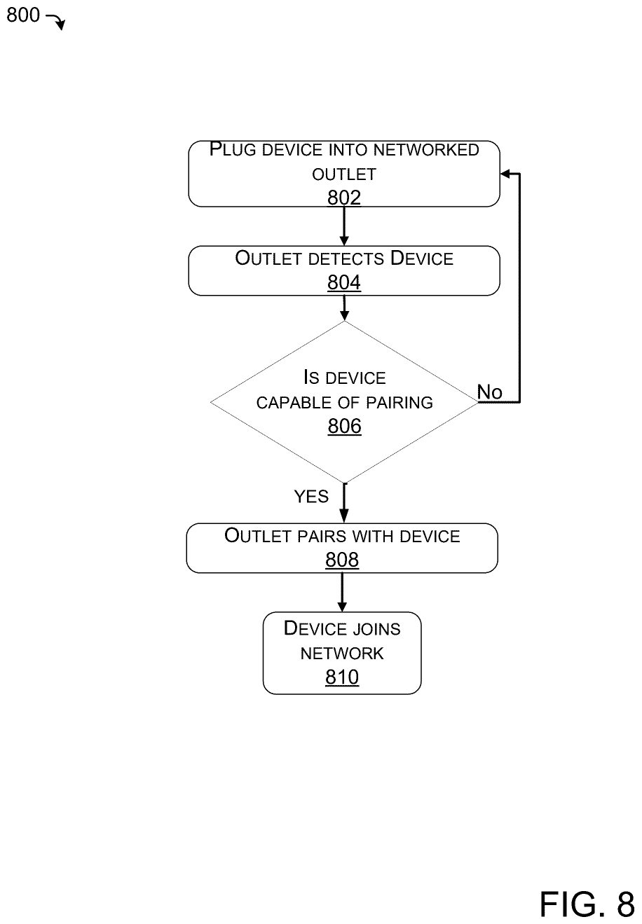

Upon detecting that a device has been plugged into the smart outlet, the smart outlet, such as smart outlet 124, may determine whether the device is capable of pairing as shown in more detail in FIG. 8. If the device is not capable of pairing, the system may act as a standard electrical outlet, providing power to the device. By plugging a network capable infusion pump 116 into outlet 124 via wired electric cord 132, a network may automatically be formed between the outlets in the patient area and the plugged in devices such as the infusion pump 116. For example, paired infusion pump 116 may exchange information with outlet 124 as shown at 126e. Outlet 125 may also communicate with the infusion pump 116 as shown at 126c and outlet 124 and outlet 125 may communicate with each other as shown at 126d.

In environment 200 of FIG. 2, the infusion pump 116 is also able to communicate with the server 102 that is in communication with RTLS 104. In other aspects, software that allows the networked devices to communicate with the RTLS 104 may be part of the RTLS 104, that is, the networked devices may be able to communicate with RTLS 104 directly without needing to communicate with an intermediate server such as server 102. Such communication may use the same or different communication protocols than the mesh network 122 shown by 126c, 126d, and 126e. The RTLS 104 and server 102 may be local or remote. In some aspects, the RTLS 104 and server 102 may be in the cloud.

In some aspects, the RTLS may be in communication with an RTLS anchor 110. Such RTLS anchors may be distributed throughout the hospital, home, or other environment. As shown in FIG. 2, the infusion pump 116 may send and receive information from the RTLS 104 via server 102 as shown by 128a and such information may then be distributed among the devices and outlets connected with the infusion pump 116 via the mesh network such as mesh network 122. Information may be sent and received as shown by 128a using any type of wireless network or other communication network known in the art including the Internet, an intranet, a wide area network (WAN), a local area network (LAN), a virtual private network (VPN), cellular network connections, and connections made using protocols such as 802.11a, b, g, n and/or ac. Alternatively or additionally, a network may include a nanoscale network, a near-field communication network, a body-area network (BAN), a personal-area network (PAN), a near-me area network (NAN), a campus-area network (CAN), and/or an inter-area network (IAN).

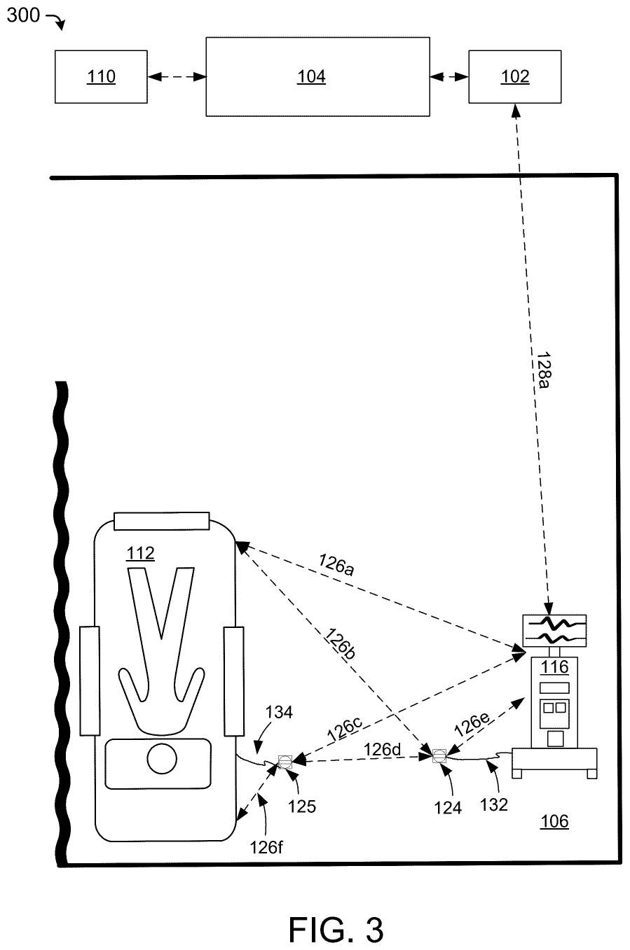

FIG. 3 depicts an environment 300 in which two devices are placed in a patient area 106, network capable bed 112 and network capable infusion pump 116. The network capable bed 112 and the network capable infusion pump 116 are able to exchange information with each other as well as to and from smart outlet 124 connected to the infusion pump via cord 132 and smart outlet 125 connected to the bed via cord 134. While patient area 106 only shows a bed 112 and an infusion pump 116 for ease of description, any number of devices may be present and connected to the mesh network 122 including the other networked devices of FIG. 1. Further, while two smart outlets are shown in FIG. 2, there is no restriction on the number of smart outlets that may be present in the patient area.

Smart outlets may have a variety of configurations that allow them to detect that an object has been plugged in via an electric cord and to determine if the object that has been plugged in is capable of joining a mesh network as described in further detail with reference to FIGS. 11 and 12. While smart outlets may have a variety of configurations, in some aspects smart outlets include a circuit board and an outlet processor configured to carry out instructions stored in an outlet memory. A wireless module enables the outlet processor to communicate with and share data with the mesh network shown at 126a-126f. The smart outlet(s) 124 and 125 may include a power receptacle for a wired electric plug and a plug detector associated with each power receptacle. In some aspects, one or more of the smart outlets may have an indicator that indicates when the outlet communication module is connected to a localized network. Other indicators or types of indicators may be used for other purposes.

Upon detecting that a device has been plugged into the smart outlet, the smart outlet may determine whether the device is capable of pairing as shown in more detail in FIG. 8. If the device is not capable of pairing, the system may act as a standard electrical outlet, providing power to the device. By plugging a network capable bed 112 into the smart outlet 125 and a network capable infusion pump into outlet 124 via wired electric cord 132 and wired electric cord 134 respectively, a network may automatically be formed between the outlets and the devices. In some aspects, paired bed 112 and infusion pump 116 may exchange information directly with each other. Similarly, the bed 112 may exchange information with outlet 124 as shown at 126b. The bed 112 may also communicate with outlet 125 as shown at 126f and outlet 125 may communicate with outlet 124 as shown at 126d. Outlet 125 may also communicate with the infusion pump 116 as shown at 126c, and outlet 124 may communicate with infusion pump 116 as shown at 126e. Communication from an outlet to an outlet, a device to a device, and an outlet to a device via a wireless mesh network may be accomplished via various network protocols including Bluetooth, ZigBee, Thread, FabFi, SMesh, and the like.

In environment 300 of FIG. 3, the infusion pump 116 is also able to communicate with the server 102 that is in communication with RTLS 104. In other aspects, software that allows the networked devices to communicate with the RTLS 104 may be part of the RTLS 104 allowing a device connected to the mesh network to communicate directly with the RTLS 104. Such communication may use the same or different communication protocols than the mesh network 122 shown by 126a-126f. The RTLS 104 and server 102 may be local or remote. In some aspects, the RTLS 104 and server 102 may be in the cloud. In some aspects, the RTLS may be in communication with an RTLS anchor 110. Such RTLS anchors may be distributed throughout the hospital, home, or other environment. Communication with the server 102 or RTLS 104 may be accomplished via various network protocols including Bluetooth, ZigBee, Thread, FabFi, SMesh, and the like.

Information may be distributed among the devices connected with the infusion pump 116 to the mesh network 122 as well as between devices and the server 102 and RTLS 104. For example, a sensor in the bed 112 containing the patient may detect movement at the edge of the bed. An alarm module may determine that the patient is getting out of the bed, triggering an alarm. However, if the bed 112 has been notified that am RTLS tag associated with a caregiver is present in the room or protocols indicate that the patient is allowed to get in and out of bed independently, the alarm module may determine that an alarm is not needed. In some aspects, instead of or in addition to notifying the systems of the bed 112, the server 102 may notify a device other than the bed 112, such as infusion pump 116 that the caregiver associated with an RTLS tag is present in the room and the infusion pump 116 may have distributed that information to the other networked devices in that room. Thus, as shown in FIG. 3, even if the bed 112 is not in direct communication with the server 102, the bed 112 may receive the information from other devices in the network and execute the appropriate protocols.

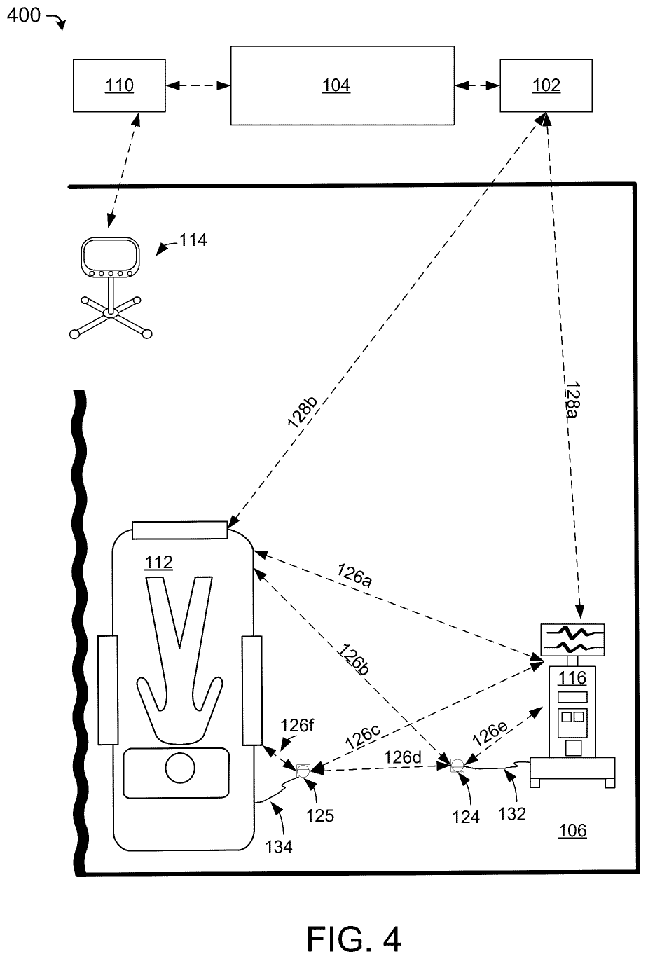

In care settings, devices may be added and removed from a room. As devices enter and leave the room, devices in the mesh network may be notified. FIG. 4 depicts the introduction of a tagged device exemplified by free-standing monitor 114 into a patient environment 400. As shown in patient environment 400 of FIG. 4, there is a pre-existing mesh network 122 between the bed 112, the infusion pump 116, and the smart outlets 124 and 125, as shown at 126a-126f. In contrast to FIG. 3, in FIG. 4 both the bed 112 and the infusion pump 116 are able to connect to the RTLS 104 via server 102 as shown by 128a and 128b respectively.

In this instance, the free-standing monitor 114 may have an RTLS tag and/or have an integrated RTLS component. For example, for an integrated RTLS component, the free-standing monitor could have an RTLS module soldered onto a printed circuit board that would be installed permanently. When the RTLS anchor 110 identifies the location of the free-standing monitor 114 as being within a patient area 106, the RTLS anchor 110 transmits a notification to the RTLS 104, the RTLS 104 communicates with the server 102 and the server 102 communicates the presence of the free-standing monitor 114 to either or both of the bed 112 as shown by 128b and the infusion pump 116 as shown by 128a. The notified device(s) may then distribute information regarding the free-standing monitor 114 to the other devices on the mesh network 122. This allows for multiple paths of communication and encourages system redundancy, decreasing the likelihood of complete communication failures.

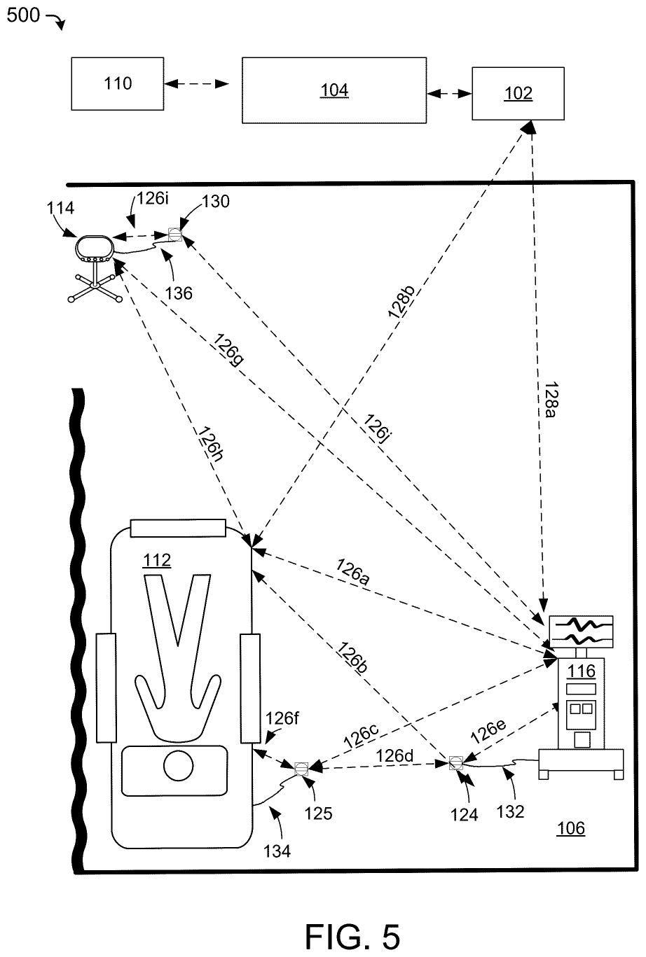

As shown in environment 500 of FIG. 5, a device, including a tagged device such as free-standing monitor 114, may be plugged into smart outlet 130 via cord 136. If the device is network capable, the device is then automatically joined to the existing mesh network 122 shown by 126a-126f. This expands the mesh network 122 to include 126g-126j. While not shown for simplicity, the smart outlet 130 may also communicate with the bed 112 and the smart outlets 124 and 125. Additionally, the free-standing monitor 114 could also communicate with smart outlets 124 and 125. The automatic addition of the new device to the mesh network 122 only requires someone to plug the device into the smart outlet via a cord such as cord 132 for the infusion pump 116, cord 134 for the bed 112 and cord 136 for the free-standing monitor 114. No further action is required on the part of the person as the smart outlet and the devices plugged into and paired to the smart outlet automatically join the existing mesh network. If the new device is not network capable, then information about the tag is communicated but the device does not join the mesh network.

Some or all of the devices connected to the mesh network may be in direct communication with the RTLS 104 or to the intermediate server 102 as shown in FIGS. 2-5. That is, some or all of the devices connected to the mesh network may be able to transmit and receive information from the RTLS 104 or intermediate server 102 without transmitting information to an intermediate networked device connected to the mesh network such as the bed 112, the free-standing monitor 114, the infusion pump 116, lighting system 118, and EKG 120. As the new device exemplified by the free-standing monitor 114 in FIG. 5 does not have an RTLS connection, free-standing monitor 114 distributes information to and receives information from the bed 112, the infusion pump 116, or both. The bed 112 and the infusion pump 116 may distribute information from the server 102 to the free-standing monitor 114 or any other devices connected to the mesh network.

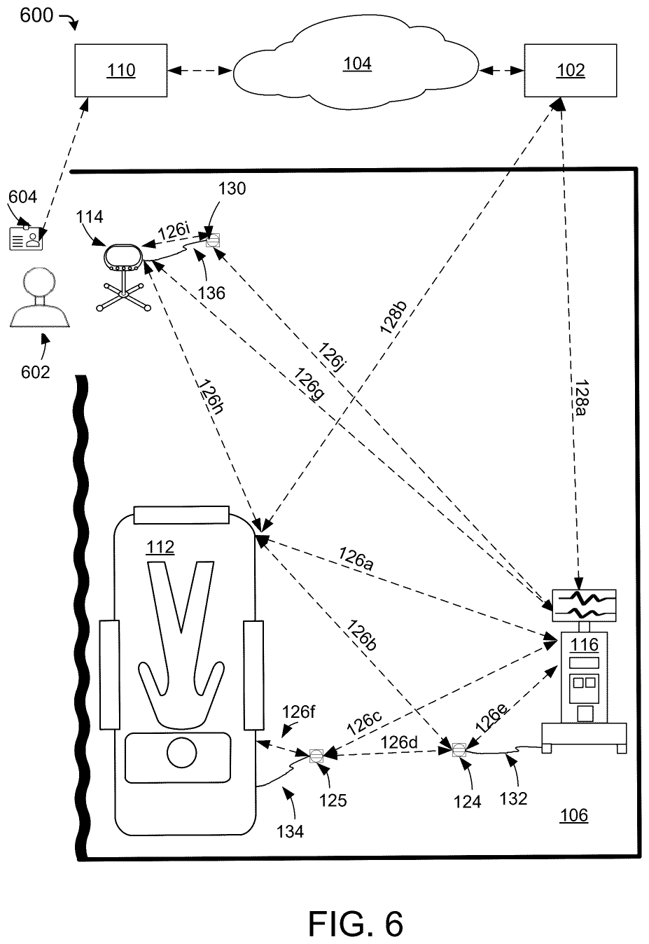

As shown in environment 600 of FIG. 6, a caregiver 602 may enter a patient area 106. The caregiver 602 may be wearing a badge 604 with an RTLS tag. The RTLS anchor 110 identifies that the badge 604 containing the RTLS tag and associated with caregiver 602 has entered the patient area 106 and notifies the RTLS 104. The RTLS 104 then transmits that notification to the server 102. The server 102 then communicates the presence of the RTLS tag in the badge 604 and the associated caregiver to one or more devices in the mesh network 122 as shown at 128a and 128b. The devices in the mesh network 122 that have received the notification then transmit that notification to the other devices connected to the mesh network 122 as shown by connections 126a-126j.

In some aspects, the entrance of the caregiver 602 wearing the badge 604 with the RTLS tag may trigger one or more protocols. For example, as the caregiver 602 enters the room, sounds and lights may change, alarms may be silenced, nurse calls may be canceled, or the type of alarm or frequency of the alarm may change. For example, audible alarms may change to visual alarms and vice versa, or the alarms may sound in a particular sequence indicating the importance of the alarm. For example, a notification that an infusion pump has finished its cycle may be less important than a patient reading indicating that the patient needs assistance. The devices in the mesh network may therefore silence alarms not related to the patient needing assistance until the issue with the patient is resolved. This assists the caregiver in prioritizing tasks and also improves the caregiver's ability to focus as they are not inundated with multiple alarms sounding at the same time. In other aspects, particular device protocols may be triggered when a tagged caregiver exits a room. That is, the RTLS notifies the mesh network 122 that the tagged caregiver 602 has exited the room. This information may be distributed throughout the mesh network 122 and protocols for each network device may determine whether particular actions need to be taken upon exit of the caregiver 602 wearing the badge 604 with the RTLS tag. For example, alarms may be primed, devices may be locked, and the like. This allows each device to make a status determination based on the information it has received from either the server 102 or from other devices within the mesh network such as mesh network 122, but does not require explicit instructions from the server 102 as protocols associated with each device determine the actions that need to occur for that device.

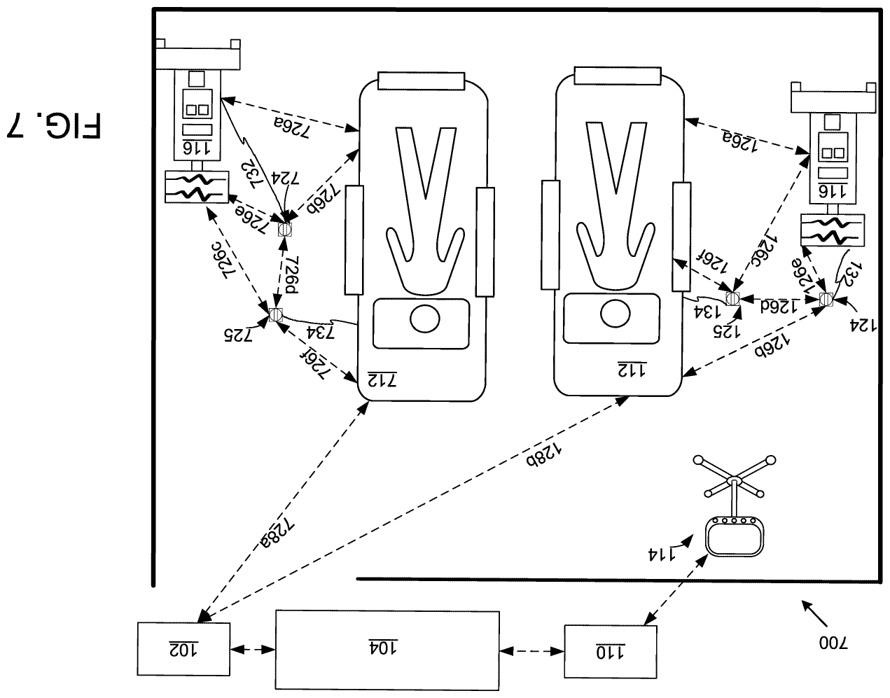

In some instances, more than one patient may occupy a patient area 106 as shown in environment 700 of FIG. 7. In this instance, there may be a plurality of mesh networks that have formed such as a network defined by connections 126a-126f associated with a first bed 112 and network defined by connections 726a-726f associated with a second bed 712 where the network defined by connections 726a-726f is similar to or the same as the network defined by connections 126a-126f but associated with a different bed or other device(s). In some aspects an application such as an application on a device such as a mobile device, tablet, or computer, may be used to group a first set of outlets for a first purpose and a second set of outlets for a second purpose. For example, there may be a first group of smart outlets associated with a first patient or portion of a patient area and a second group of smart outlets associated with a second patient or portion of a patient area. Thus, when a device is plugged into a smart outlet such as smart outlet 725 or smart outlet 724 using cord 732 or cord 734, respectively, where smart outlet 725 and smart outlet 724 are associated with a second patient or second area, the device joins the second network 726a-726f and does not interact with the first network defined by connections 126a-126f formed among devices such as bed 112 and infusion pump 116 and smart outlet 124 or smart outlet 125. As shown in FIG. 7, information transmitted to bed 712, that is information associated with mesh network 726a-726f is transmitted via signal 728a to server 102, and information transmitted to bed 112 from network defined by connections 126a-126f is transmitted to server 102 via signal 128b.

A mesh network such as mesh network 122 may be formed between a plurality of smart outlets and devices within a patient area such as patient area 106 as shown in process 800 of FIG. 8. A device may be plugged into a smart outlet at 802. The smart outlet detects the device at 804. At 806, the smart outlet determines whether the device is capable of pairing at 806. If the device is capable of pairing, the outlet pairs with the device at 808 and the device is connected to or forms a mesh network at 810. If the device is not capable of pairing at 806, the smart outlet behaves like a regular outlet and waits for another device to be plugged in at 802. In some aspects, the systems and methods described in U.S. patent application Ser. No. 17/577,496, published as U.S. Patent Application Publication No. 2022/0233382 A1 may be used for creating a secured one-to-one connection.

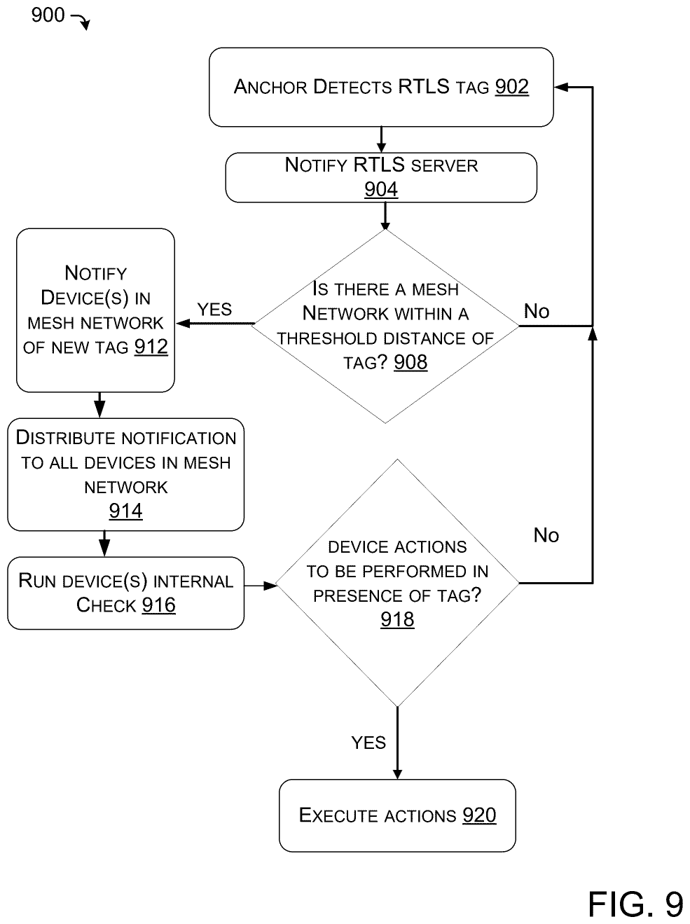

Process 900 of FIG. 9 depicts a scenario such as the scenario shown in FIG. 4 and FIG. 6 in which a new tagged device or caregiver enters the patient area 106. The RTLS tag such as tag 108 is detected at 902 by an anchor such as anchor 110. The anchor sends a signal that reaches the RTLS at 904. The RTLS such as RTLS 104 then notifies a server such as server 102 as shown in FIG. 1. The server then determines if there is a mesh network within a threshold distance of the tagged caregiver or device at 908. Such a threshold distance may be any distance generally determined for a patient area such as patient area 106 and may be the same or different for each patient area. In some aspects, the threshold may be pre-set based on the type of signaling being used. In other aspects, the threshold may be adjusted based on the configuration of a particular patient environment or within a particular clinical setting. In some aspects, the tagged caregivers or devices may be identified as being within a specific distance from a second device or from a smart outlet. If the server determines that the RTLS tag location is within a threshold distance from the mesh network, the server may notify one or more devices in the mesh network of the presence of the new RTLS tagged device at 912 as shown, for example in FIG. 4. The device(s) receiving the notification then distributes the notification to some or all of the other devices within the mesh network at 914. In some aspects, there may be pre-defined actions that take place when an additional device is added to a patient area. For example, if a caregiver with an RTLS tag enters a room, a tv may be turned off, lights may be brightened, alarms may be silenced, and the like. Each device in the mesh network may run an internal check at 916 to determine if there are actions to be taken at 918. If there are actions triggered by the presence of the device or caregiver associated with the RTLS tag, they may be executed at 920. If there are no actions to be taken at 918, the system waits until a new RTLS tag or location change of the existing RTLS tag is detected at 902.

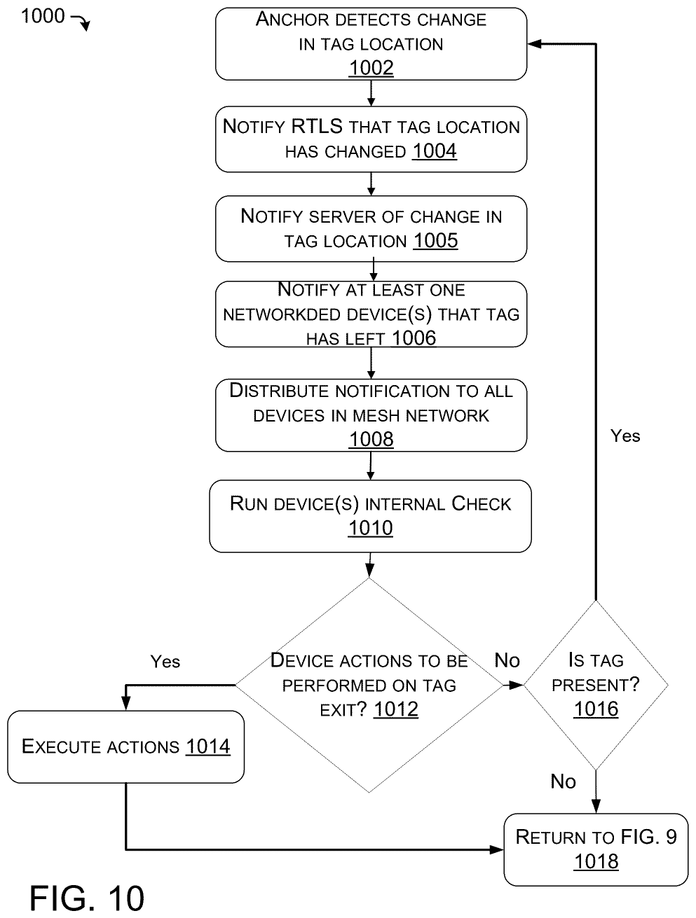

Process 1000 of FIG. 10 depicts the actions that take place when a tagged device or caregiver exits a patient area. As shown in FIG. 10, the RTLS anchor may detect when the RTLS tag moves from a patient area by more than a threshold amount at 1002. The RTLS anchor may then notify the RTLS 104 at 1004. The RTLS then notifies a server such as server 102 at 1005. The server then notifies one or more networked devices that the tag has left at 1006. The networked devices then distribute the notification to some or all of the devices in the mesh network at 1008. Each networked device then runs an internal check at 1010 to determine if there are specific actions that should be performed at 1012. For example, when the tag leaves, alarms may reset, light and sound settings may change, certain networked devices may lock, and the like. The devices then execute the actions at 1014 and return to FIG. 9 at 1018 to wait for another tag. If there are no actions to be performed at 1012, the networked devices check for other tags still in the patient area at 1016 and then wait for notification that one or more of the other tags have changed location at 1016.

Referring now to FIG. 11, a system 1100 for automatically provisioning devices in a localized wireless network includes at least one smart outlet such as smart outlet 1125 similar to smart outlet 124, smart outlet 125, or smart outlet 130. The smart outlet 1125 includes a circuit board 1006. In the illustrated embodiment, the circuit board 1106 is a system-on-module (SOM) circuit board. The circuit board 1006 includes an outlet processor 1108 configured to carry out instructions stored in an outlet memory 1112. A wireless module 1114 enables the outlet processor 1108 to communicate with and share data with a facility network 1142 including an RTLS. An outlet communication module 1116 enables the outlet processor 1108 to communicate with and share data with other networked devices, as described in more detail below. In some embodiments, the outlet communication module 1116 is a Bluetooth module. The smart outlet 1125 includes at least one power receptacle such as power receptacle 1102a for a plug and a plug detector 1104 associated with each power receptacle exemplified by power receptacle 1102. Although the illustrated embodiment shows two power receptacles 1102a and 1102b, it will be appreciated that the smart outlet 1125 includes any number of power receptacles 1102 and associated plug detectors such as plug detector 1104a and plug detector 1104b, in some embodiments. Each of the power receptacles 1102 is in communication with the circuit board 1106. In some embodiments, the power receptacles 1102 include an alternating current power receptacle. The plug detectors such as 1104a and 1104b detect a plug inserted into the associated power receptacle 1102 and transmits a signal to the outlet processor 1108 to begin a provisioning routine, as described in more detail in FIG. 8. A timer 1118 is started to measure an uptime in response to a power plug being plugged into a power receptacle 1102 as detected by the plug detector 1104.

The system 1100 is configured to provision at least one medical device 1120 creating a networked device in communication with a server in communication with an RTLS. The medical device 1120 includes a circuit board 1124. In the illustrated embodiment, the circuit board 1124 is a master control board (MCB) board. The circuit board 1124 includes a device processor 1126 that carries out instructions stored in a memory 1128. A wireless module 1130 enables the device processor 1126 to communicate with and share data with the facility network 1142. A timer 1132 measures an uptime in response to sensing that the medical device 1120 is receiving power. A power plug 1122 is configured to insert into one of the power receptacles 1102 of the smart outlet 1125. A communication circuit board 1138 includes a communication processor 1134 that carries out instructions stored in a memory 1136. The circuit board 1138 includes a device communication module 1140 that is configured to communicate with the outlet communication module 1116 so that the medical device 1120 and the smart outlet 1125 can share data. In some embodiments, the device communication module 1140 is a Bluetooth module.

Referring now to FIG. 12, the system 1200 automatically provisions devices. The smart outlet 1225 similar to smart outlet 124, smart outlet 125, and smart outlet 130, includes a circuit board 1206. In the illustrated embodiment, the circuit board 1206 is a system-on-module (SOM) circuit board. The circuit board 1206 includes an outlet processor 1208 configured to carry out instructions stored in an outlet memory 1212. A wireless module 1214 enables the outlet processor 1208 to communicate with and share data with a facility network 1232. An outlet communication module 1216 enables the outlet processor 1208 to communicate with and share data with other networked devices, as described in more detail below. In some embodiments, the outlet communication module 1216 is a Bluetooth module. The smart outlet 1225 includes at least one power receptacle for a plug and a plug detector associated with each power receptacle. Although the illustrated embodiment shows two power receptacles 1202a and 1202b, it will be appreciated that the smart outlet 1225 includes any number of power receptacles and associated plug detectors such as plug detectors 1204a and 1204b, in some embodiments. Each of the power receptacles 1202a and 1202b is in communication with the circuit board 1206. In some embodiments, the power receptacles such as power receptacle 1202a and power receptacle 1202b include an alternating current power receptacle. The plug detectors 1204a or 1204b detect a plug inserted into the associated power receptacle 1202a or 1202b and transmits a signal to the outlet processor 1208 to begin a provisioning routine, as described in more detail in FIG. 8. A timer 1218 is started to measure an uptime in response to a power plug being plugged into a power receptacle 1202a or 1202b as detected by the plug detector 1204a or 1204b.

The system 1200 is configured to provision at least one medical device 1220, similar to the medical devices shown in FIG. 1, though other medical devices are also contemplated, creating a networked device. A power plug 1222 of the medical device 1220 is configured to insert into one of the power receptacles 1202a or 1202b of the smart outlet 1225. The medical device 1220 includes a circuit board 1224. In the illustrated embodiment, the circuit board 1224 is a communication module. The circuit board 1224 includes a device processor 1226 that carries out instructions stored in a memory 1228. A Bluetooth module 1230 allows the medical device to communicate with other medical devices in the mesh network such as the medical device of FIG. 11, allowing the processor 1226 to communicate with and share data with the medical device 1120 of FIG. 11. It may also allow the medical device 1120 and/or the medical device 1220 to communicate with outlet communication module 1216 so that the medical device(s) and the smart outlet 125 can share data.

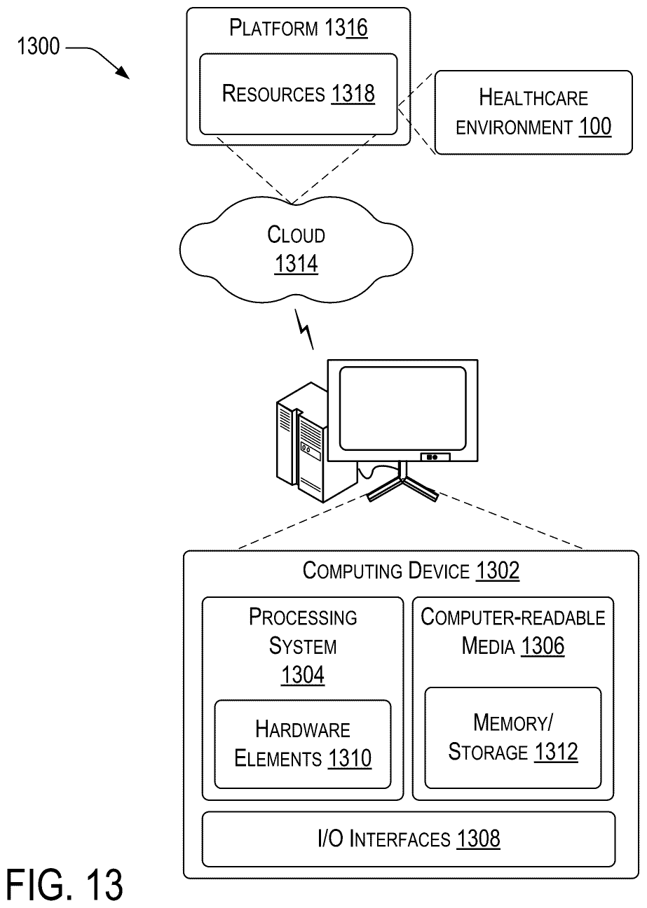

FIG. 13 illustrates an example system generally at 1300 that includes a computing device 1302 that is representative of one or more computing systems and/or devices that may implement the various techniques, flowcharts, and modules described herein. This is illustrated through the inclusion of the health care environment 100, though this may include the other systems and environments as shown for example in FIGS. 2-7. The computing device 1302 may be, for example, a server of a service provider such as a server in an RTLS, an intermediate server such as server 102, a hospital system, a device associated with a client (e.g., a client device), an on-chip system, and/or any other suitable computing device or computing system.

The computing device 1302 as illustrated includes a processing system 1304, one or more computer-readable media 1306, and one or more I/O interface 1308 that are communicatively coupled, one to another. In some embodiments, the processor(s) of the processing system includes a Central Processing Unit (CPU), a Graphics Processing Unit (GPU), or both CPU and GPU, or other processing unit or component known in the art. Although not shown, the computing device 1302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1304 is representative of the functionality used to perform one or more operations using hardware. Accordingly, the processing system 1304 is illustrated as including hardware element 1310 that may be configured as processors, functional blocks, and so forth. This may include implementation in hardware as an application specific integrated circuit or other logic device formed using one or more semiconductors. The hardware elements 1310 are not limited by the materials from which they are made, or the processing mechanisms employed therein. For example, processors may include semiconductor(s) and/or transistors (e.g., electronic integrated circuits (ICS)). In such a context, processor-executable instructions may be electronically executable instructions.

The computer-readable media 1306 is illustrated as including memory/storage component 1312. The memory/storage component 1312 represents memory/storage capacity associated with one or more computer-readable media. The memory/storage component 1312 may include volatile media (such as random-access memory (RAM)) and/or nonvolatile media (such as read-only memory (ROM), Flash memory, optical disks, magnetic disks, and so forth). The memory/storage component 1312 may include fixed media (e.g., RAM, ROM, a fixed hard drive) as well as removable media (e.g., Flash memory, a removable hard drive, an optical disc). The computer-readable media 1306 may be configured in a variety of other ways as further described below.

I/O interface 1308 (Input/Output interface) is representative of functionality to allow a user to input commands and information to computing device 1302, and also to allow information to be presented to the user and/or other components or devices using various input/output devices. Examples of input devices include a keyboard, a cursor control device (e.g., a mouse or touch pad), a microphone, a scanner, touch screen (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1302 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” “logic,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on and/or transmitted across some form of computer-readable media. The computer-readable media may include a variety of media that may be accessed by the computing device 1302. By way of example, and not limitation, computer-readable media may include “computer-readable storage media” and “computer-readable transmission media.”

“Computer-readable storage media” may refer to media and/or devices that enable persistent and/or non-transitory storage of information in contrast to mere signal transmission, carrier waves, or signals per se. Thus, computer-readable storage media refers to non-signal-bearing media. The computer-readable storage media includes hardware such as volatile and non-volatile, removable and non-removable media, and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable transmission media” may refer to a medium that is configured to transmit instructions to the hardware of the computing device 1302, such as via a network. Computer-readable transmission media typically may transmit computer-readable instructions, data structures, program modules, or other data in a modulated data signal, such as carrier waves, data signals, or other transport mechanisms. Computer-readable transmission media also include any information delivery media. The term “modulated data signal” means a signal that has one or more of its characteristics set or changed in such a manner as to encode information in the signal. By way of example, and not limitation, computer-readable transmission media include wired media such as a wired network or direct-wired connection, and wireless media such as acoustic, radio frequency (RF), infrared, and other wireless media.

As previously described, hardware elements 1310 and computer-readable media 1306 are representative of modules, programmable device logic, and/or device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1310. The computing device 1302 may be configured to execute particular instructions and/or functions corresponding to the software and/or hardware modules. Accordingly, implementation of a module that is executable by the computing device 1302 as software may be achieved at least partially in hardware, e.g., through the use of computer-readable storage media and/or hardware elements 1310 of the processing system 804. The instructions and/or functions may be executable/operable by one or more articles of manufacture (for example, one or more computing devices 1302 and/or processing systems 1304) to implement techniques, modules, and examples described herein.

The techniques described herein may be supported by various configurations of the computing device 1302 and are not limited to the specific examples of the techniques described herein. This functionality may also be implemented all or in part through the use of a distributed system, such as over a “cloud” 1314 via a platform 1316 as described below.

The cloud 1314 includes and/or is representative of a platform 1316 for resources 1318. Platform 1316 abstracts the underlying functionality of hardware (e.g., servers) and software resources of the cloud 1314. The resources may be accessed or distributed using any type of wireless network or other communication network known in the art. Examples include the Internet, an intranet, a wide area network (WAN), a local area network (LAN), a virtual private network (VPN), cellular network connections, and connections made using protocols such as 802.11a, b, g, n and/or ac. Alternatively or additionally, a network may include a nanoscale network, a near-field communication network, a body-area network (BAN), a personal-area network (PAN), a near-me area network (NAN), a campus-area network (CAN), and/or an inter-area network (IAN).

The resources 1318 may include applications and/or data that can be utilized while computer processing is executed on servers that are remote from the computing device 1302. Resources 1318 can also include services provided over the Internet and/or through a subscriber network, such as a cellular or Wi-Fi network.

Platform 1316 may abstract resources and functions to connect the computing device 802 with other computing devices. The platform 1316 may also be scalable to provide a corresponding level of scale to encountered demand for the resources 1318 that are implemented via the platform 1316. Accordingly, in an interconnected device embodiment, implementation of functionality described herein may be distributed throughout multiple devices of the system 1300. For example, the functionality may be implemented in part on the computing device 1302 as well as via the platform 1316 which may represent a cloud computing environment.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as examples only, with a true scope and spirit of the present disclosure being indicated by the following claims.

### EXAMPLE CLAUSES

1. A system including: a first medical device having a first communication module and a first wired electric plug; a second medical device having a second communication module and a second wired plug; at least a first outlet and a second outlet, each of the first outlet and the second outlet including: a power receptacle; and a plug detector in communication with a respective outlet processor and a respective outlet communication module, wherein when the first medical device and the second medical device is plugged into the first outlet or the second outlet, a mesh network is formed; a real time locating system (RTLS); an RTLS anchor in communication with the RTLS; an RTLS tag associated with a third device and detectable by the RTLS anchor; and a server in communication with the RTLS, wherein the RTLS anchor is configured to transmit a location of the RTLS tag to the RTLS, and based on receiving the location from the RTLS anchor, the RTLS is configured to transmit the location to the server, and based on receiving the location from the RTLS, the server is configured to transmit the location of the RTLS tag to at least one of the first medical device or the second medical device.

2. The system of clause 1, wherein the first medical device and the second medical device can communicate with any other medical device in the mesh network.

3. The system of clauses 1 or 2, wherein: prior to the server transmitting the location of the RTLS tag to at least one of the first medical device and the second medical device, the server is configured to determine that the RTLS tag is disposed within a threshold distance of the mesh network.

4. The system of any of clauses 1 to 3, wherein the third device is a medical device or a badge of a caregiver.

5. The system of any of clauses 1 to 4, wherein the respective outlet communication module communicates with the respective device communication module of each of the first medical device and the second medical device.

6. The system of any of clauses 1 to 5, wherein each outlet communication module is wireless.

7. The system of any of clauses 1 to 6, further including a fourth medical device with a fourth device communication module and a fourth wired plug plugged into a third outlet.

8. The system of clause 6, wherein each medical device in the mesh network can communicate with every other medical device in the mesh network.

9. The system of clause 8, wherein the server sends and receives information from only one medical device of at least the first medical device and the second medical device.

10. The system of clause 9, wherein the one medical device transmits information from the other medical devices of at least the first medical device and the second medical device to the server.

11. The system of clause 8, wherein the server transmits and receives information from at least two medical devices of at least the first medical device and the second medical device.

12. A method of automatically initiating workflows including: receiving from a server, by a first medical device of a plurality of medical devices, an indication that an RTLS tag is within a threshold distance of a mesh network, the mesh network including the plurality of medical devices and a plurality of power receptacles, each power receptacle of the plurality of power receptacles being: configured to provide power, via a cord, to a medical device of the plurality of medical devices, and associated with a wireless communicator configured to wirelessly communicate with each medical device of the plurality of medical devices; transmitting from the first medical device to the plurality of medical devices via the mesh network, the indication of the RTLS tag within a threshold distance; determining, by each medical device of the plurality of medical devices, an action to be taken by the respective medical device based on the indication of the RTLS tag within the threshold distance of the mesh network; and executing, by each medical device of the plurality of medical devices, the action to be taken.

13. The method of clause 12, wherein the server determines that the RTLS tag is within a threshold distance is within the mesh network.

14. The method of clauses 12 or 13, wherein the RTLS tag is attached to a caregiver.

15. The method of any of clauses 12 to 14, wherein the action permits the caregiver associated with the RTLS tag access to settings of a medical device of the plurality of medical devices.

16. The method of any of clauses 12 to 14, wherein the action restricts access by the caregiver associated with the RTLS tag to settings of a medical device of the plurality of medical devices.

17. A system network including: a real time locating system (RTLS) containing a first processor; an RTLS anchor containing a second processor and in communication with the RTLS; an RTLS tag detectable by the RTLS; a server containing a third processor and in communication with the RTLS; and a mesh network including: a first medical device containing a fourth processor; a first power receptacle configured to provide power via a first cord to the first medical device, the first power receptacle being associated with a wireless communicator to provide wireless communication between the first power receptacle and the first medical device, a second medical device containing a fifth processor; and a second power receptacle configured to provide power via a second cord to the second medical device, the second power receptacle being associated with a second wireless communicator to provide wireless communication between the second power receptacle and the second medical device; wherein the RTLS anchor is configured by the second processor to track a location RTLS tag and transmit the location and identity of the RTLS tag to the RTLS, and the RTLS is configured by the first processor to communicate wirelessly with third processor of the server and the third process or of the server is configured to communicate with at least one of the fourth processor of the first medical device and the fifth processor of the second medical device to provide information about the location of the RTLS tag received from the RTLS anchor.

18. The system network of clause 17, wherein the medical device in receipt of the information about the RTLS tag distributes the information to the other medical device.

19. The system network of embodiments 17 or 18, wherein the RTLS tag is associated with a caregiver.

20. The system network of any of clauses 17 to 19, wherein the processor of at least one device of the first medical device and the second medical device acts on information about the RTLS tag.

21. The system network of any of clauses 17 to 19, wherein the RTLS tag is associated with a third medical device.

22. A system including: at least a first medical device, wherein each device includes a device communication module and a wired plug; at least a first outlet and a second outlet, each of the first outlet and the second outlet including: a power receptacle; and a plug detector in communication with a respective outlet processor and a respective outlet communication module, wherein when the first medical device is plugged into the first outlet or the second outlet, a mesh network is formed between the first medical device, the first outlet and the second outlet; a real time locating system (RTLS); an RTLS anchor in communication with the RTLS; an RTLS tag associated with a third device and detectable by the RTLS anchor; and a server in communication with the RTLS, wherein the RTLS anchor is configured to transmit a location of the RTLS tag to the RTLS, and based on receiving the location from the RTLS anchor, the RTLS is configured to transmit the location to the server, and based on receiving the location from the RTLS, the server is configured to transmit the location of the RTLS tag to the at least one medical device or the first outlet or second outlet; wherein the first medical device can communicate with any other medical device or smart outlet in the mesh network.

23. The system of clause 22, wherein: prior to the server transmitting the location of the RTLS tag to the first medical device or the smart outlet, the server is configured to determine that the RTLS tag is disposed within a threshold distance of the mesh network.

24. The system of clause 23, wherein the third device is a second medical device or a badge of a caregiver.

25. The system of any of clauses 22 to 24, wherein the server sends and receives information from only one of the first medical device, the first outlet, or the second outlet.

26. The system of any of clause 25, wherein the first medical device receives information from the server and sends the information to the first outlet and the second outlet.

27. A system including: at least a first medical device and a second medical device, wherein each device includes a device communication module and a wired electric plug, wherein when the first medical device and the second medical device are plugged in, a mesh network is formed; a real time locating system (RTLS); an RTLS anchor in communication with the RTLS; an RTLS tag associated with a third device and detectable by the RTLS anchor; and a server in communication with the RTLS, wherein the RTLS anchor is configured to transmit a location of the RTLS tag to the RTLS, and based on receiving the location from the RTLS anchor, the RTLS is configured to transmit the location to the server, and based on receiving the location from the RTLS, the server is configured to transmit the location of the RTLS tag to at least one of the first medical device and the second medical device; wherein the first medical device and the second medical device can communicate with any other medical device in the mesh network.

28. The system of clause 27, further including at least a first outlet and a second outlet, each of the first outlet and the second outlet including: a power receptacle; and a plug detector in communication with a respective outlet processor and a respective outlet communication module, wherein the device is plugged into the first outlet or the second outlet.

29. The system of clause 28, wherein: prior to the server transmitting the location of the RTLS tag to at least one of the first medical device and the second medical device, the server is configured to determine that the RTLS tag is disposed within a threshold distance of the mesh network.

30. The system of clause 28 or 29, wherein the third device is a medical device or a badge of a caregiver.

32. The system of any of clauses 28 to 31, wherein the respective outlet communication module communicates with the respective device communication module of each of the first medical device and the second medical device.

33. The system of any of clauses 29 to 31, wherein each outlet communication module is wireless.

34. The system of any of clauses 29 to 31, further including a fourth medical device with a fourth device communication module and a fourth wired plug plugged into a third outlet.

35. The system of clause 33, wherein each medical device in the mesh network can communicate with every other medical device in the mesh network.

36. The system of clause 34, wherein the server sends and receives information from only one medical device of at least the first medical device and the second medical device.

37. The system of clause 35, wherein the one medical device transmits information from the other medical devices of at least the first medical device and the second medical device to the server.

38. The system of any of clauses 33 to 36, wherein the server transmits and receives information from at least two medical devices of at least the first medical device and the second medical device.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components unless the context requires otherwise.

As used herein, the term “based on” can be used synonymously with “based, at least in part, on” and “based at least partly on.”

As used herein, the terms “comprises/comprising/comprised” and “includes/including/included,” and their equivalents can be used interchangeably. An apparatus, system, or method that “comprises A, B, and C” includes A, B, and C, but also can include other components (e.g., D) as well. That is, the apparatus, system, or method is not limited to components A, B, and C.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and website content throughout this specification (referenced materials herein). Each of the referenced materials is individually incorporated herein by reference in their entirety for their referenced teaching(s), as of the filing date of this application.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when the application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art.

Although the subject matter has been described in language specific to structural features and/or methodological acts, it is to be understood that the subject matter defined in the appended claims is not necessarily limited to the specific features or acts described.

What is claimed is:

1. A system comprising:
a first medical device having a first communication module and a first wired electric plug;
a second medical device having a second communication module and a second wired plug;
at least a first outlet and a second outlet, each of the first outlet and the second outlet including:
a power receptacle; and
a plug detector in communication with a respective outlet processor and a respective outlet communication module, wherein when the first medical device and the second medical device is plugged into the first outlet or the second outlet, a mesh network is formed;
a real time locating system (RTLS);
an RTLS anchor in communication with the RTLS;
an RTLS tag associated with a third device and detectable by the RTLS anchor; and
a server in communication with the RTLS,
wherein the RTLS anchor is configured to transmit a location of the RTLS tag to the RTLS, and
based on receiving the location from the RTLS anchor, the RTLS is configured to transmit the location to the server, and
based on receiving the location from the RTLS, the server is configured to transmit the location of the RTLS tag to at least one of the first medical device or the second medical device.

2. The system of claim 1, wherein the first medical device and the second medical device can communicate with any other medical device in the mesh network.

3. The system of claim 1, wherein:
prior to the server transmitting the location of the RTLS tag to at least one of the first medical device and the second medical device, the server is configured to determine that the RTLS tag is disposed within a threshold distance of the mesh network.

4. The system of claim 3, wherein the third device is a medical device or a badge of a caregiver.

5. The system of claim 1, wherein the respective outlet communication module communicates with the respective device communication module of each of the first medical device and the second medical device.

6. The system of claim 1, wherein each outlet communication module is wireless.

7. The system of claim 1, further comprising a fourth medical device with a fourth device communication module and a fourth wired plug plugged into a third outlet.

8. The system of claim 6, wherein each medical device in the mesh network can communicate with every other medical device in the mesh network.

9. The system of claim 8, wherein the server sends and receives information from only one medical device of at least the first medical device and the second medical device.

10. The system of claim 9, wherein the one medical device transmits information from the other medical devices of at least the first medical device and the second medical device to the server.

11. The system of claim 8, wherein the server transmits and receives information from at least two medical devices of at least the first medical device and the second medical device.

12. A method of automatically initiating workflows comprising:
receiving from a server, by a first medical device of a plurality of medical devices, an indication that an RTLS tag is within a threshold distance of a mesh network, the mesh network comprising the plurality of medical devices and a plurality of power receptacles, each power receptacle of the plurality of power receptacles being:
configured to provide power, via a cord, to a medical device of the plurality of medical devices, and
associated with a wireless communicator configured to wirelessly communicate with each medical device of the plurality of medical devices;
transmitting from the first medical device to the plurality of medical devices via the mesh network, the indication of the RTLS tag within a threshold distance;
determining, by each medical device of the plurality of medical devices, an action to be taken by the respective medical device based on the indication of the RTLS tag within the threshold distance of the mesh network; and
executing, by each medical device of the plurality of medical devices, the action to be taken.

13. The method of claim 12, wherein the server determines that the RTLS tag is within a threshold distance is within the mesh network.

14. The method of claim 12, wherein the RTLS tag is attached to a caregiver.

15. The method of claim 14, wherein the action permits the caregiver associated with the RTLS tag access to settings of a medical device of the plurality of medical devices.

16. The method of claim 14, wherein the action restricts access by the caregiver associated with the RTLS tag to settings of a medical device of the plurality of medical devices.

17. A system network comprising:
a real time locating system (RTLS) containing a first processor;
an RTLS anchor containing a second processor and in communication with the RTLS;
an RTLS tag detectable by the RTLS;
a server containing a third processor and in communication with the RTLS; and
a mesh network comprising:
a first medical device containing a fourth processor;
a first power receptacle configured to provide power via a first cord to the first medical device, the first power receptacle being associated with a wireless communicator to provide wireless communication between the first power receptacle and the first medical device,
a second medical device containing a fifth processor; and
a second power receptacle configured to provide power via a second cord to the second medical device, the second power receptacle being associated with a second wireless communicator to provide wireless communication between the second power receptacle and the second medical device;
wherein the RTLS anchor is configured by the second processor to track a location RTLS tag and transmit the location and identity of the RTLS tag to the RTLS, the RTLS is configured by the first processor to communicate wirelessly the server, and the server is configured by the third processor to communicate wirelessly with at least one of the fourth processor of the first medical device and the fifth processor of the second medical device to provide information about the location of the RTLS tag received from the RTLS anchor.

18. The system network of claim 17, wherein the medical device in receipt of the information about the RTLS tag distributes the information to the other medical device.

19. The system network of claim 17, wherein the RTLS tag is associated with a caregiver.

20. The system network of claim 19, wherein the processor of at least one device of the first medical device and the second medical device acts on information about the RTLS tag.
